(19) United States
(12) Patent Application Publication
(10) Pub. No.: US 2024/0207337 A1
(43) Pub. Date: Jun. 27, 2024

(54) MYCELIAL CULTURE OF Irpex lacteus AND COMPOSITION COMPRISING SAME AS ACTIVE INGREDIENT FOR PREVENTION AND TREATMENT OF DIABETES MEILLITUS

(71) Applicants: Byoung Cheon kim, Gimcheon-si, Gyeongsangbuk-do (KR); Jianmin LI, Singapore (SG); LONGRUNN MEDICAL FOOD PTE. LTD., Singapore (SG)

(72) Inventors: Byoung Cheon KIM, Gimcheon-si, Gyeongsangbuk-do (KR); Jianmin LI, Singapore (SG)

(73) Assignees: Byoung Cheon KIM, Gimcheon-si, Gyeongsangbuk-do (KR); Jianmin LI, Singapore (SG); LONGRUNN MEDICAL FOOD PTE. LTD., Singapore (SG)

(21) Appl. No.: 18/012,533

(22) PCT Filed: Jun. 8, 2022
(86) PCT No.: PCT/KR2022/008072

§ 371 Date: Mar. 18, 2024

(30) Foreign Application Priority Data
Jun. 15, 2021 (KR) 10-2021-0077526

(51) Int. Cl. A61K 36/07; A23L 31/00; A61P 5/50; C12N 1/14

(52) U.S. Cl. CPC A61K 36/07; A23L 31/00; A61P 5/50; C12N 1/14

(57) ABSTRACT

The present disclosure relates to a mycelial culture of Irpex lacteus and a composition comprising same as an active ingredient for prevention and treatment of diabetes mellitus. The mycelial culture of Irpex lacteus is expected to find advantageous applications in preventing and alleviating diabetes mellitus as it was found to have high contents of extracellular polysaccharides and beta-glucan which are prophylactically and therapeutically effective for diabetes mellitus and safe as a natural substances and to exhibit an excellent anti-diabetic effect.

### CROSS REFERENCE TO RELATED APPLICATION

This application is a 35 U.S.C. 371 National Phase Entry Application from PCT/KR2022/008072, filed on Jun. 8, 2022, which claims the benefit of Korean Patent Application No. 10-2021-0077526 filed on Jun. 15, 2021, the disclosures of which are incorporated by reference herein in their entirety.

The present application includes a Sequence Listing filed in electronic format. The Sequence Listing is entitled “3738-194_ST25.txt” created on Mar. 18, 2024 and is 4,181 bytes in size. The information in the electronic format of the Sequence Listing is part of the present application and is incorporated herein by reference in its entirety.

### TECHNICAL FIELD

The present disclosure relates to an Irpex lacteus culture and a composition containing same as an active ingredient for prevention and treatment of diabetes mellitus.

### BACKGROUND ART

Diabetes mellitus is one of representative chronic adult diseases, and the number of patients therewith in Korea is increasing every year with the improvement of living standards and the westernization of lifestyle. Diabetes mellitus is a metabolic disease characterized by a high blood glucose level which may result from an impairment in the cellular potential of normally utilizing glucose and by excretion of glucose in the urine.

Diabetes mellitus differs in cause depending on types of disease and is generally caused by a combination of genetic and environmental traits. Mechanisms accounting for hyperglycemia includes insulin secretion impairment, glucose metabolism impairment in peripheral tissues, and excessive generation of glucose in the liver. There are two main types of diabetes mellitus: type 1 diabetes and type 2 diabetes. Type 1 diabetes results from failure of the pancreas to produce enough insulin due to loss of beta cells. Type 2 diabetes is characterized by insulin resistance, together with various degrees of the functional impairment of beta cells. Disruption of multiple glucose metabolic pathways in diabetes may lead to complications associated with coronary artery disease and stroke as well as various microvascular complications in the kidney, the retina, and the nerves.

The most common causes of diabetes include excessive food intake, lack of exercise, and genetic susceptibility. Diabetes has negative consequences for health and quality of life, such as type 2 diabetes, hypertension, hypercholesterolemia, and cardiovascular disease, with social costs therefor gradually increasing. Diabetes is the second leading cause of preventable deaths in the United States with over 300,000 deaths annually. The resulting medical and social costs are reported to exceed $68 billion each year.

As such, diabetes causes various metabolic diseases such as dyslipidemia, increased oxidative stress resulting from production of free radicals, lipid peroxidation, atherosclerosis, etc. and aggravates the severity thereof. Thus, there is an urgent need for prophylactic or therapeutic methods for diabetes. To date, drugs for controlling blood sugar levels in type 2 diabetes patients are commercially available as oral dosage formulations. Representative of the oral agents are sulfonylureas, biguanides, alpha-glucosidase inhibitors, thiazolidinediones, and meglitinides. However, when used for a long time, the GLP-1 antidiabetic agents such as Byetta, Zanuvia, etc., acts on the exocrine pancreas to unnecessarily promote the proliferation of pancreatic ductal cells and thus partially block the passage of digestive enzymes, causing local pancreatic inflammation. In fact, long-term administration of such antidiabetic agents raises concerns about the side effects of pancreatitis and pancreatic cancer, with a six-fold higher risk found for pancreatitis. Accordingly, there is an urgent need for development of a natural material-based antidiabetic agent free of adverse effects.

Irpex lacteus is a common crust fungus distributed throughout temperature areas of the world. According to the scientific classification, it belongs to the genus Irpex, the family Meruliaceae, the order Polyporales, the class Agaricomycetes, the division Basidiomycota. Irpex lacteus is found in South Korea (Nam Mountain, Sobaek Mountain, Odae Mountain, Gaya Mountain, Duryun Mountain, and Chiri Mountain, and the Byeonsanbando National Part), North Korea (Paektu Mountain), China, Siberia, Europe, and North America, and is a non-resupinate mushroom.

Irpex lacteus, which is a kind of wood-rotting fungi inhabiting mainly oaks, degrades woods into inorganic materials. With excellent biological degradation ability for chemically synthesized, environmental pollutants, industrial wastes, and explosive materials, the fungus is utilized for environmental purification. In addition, Irpex lacteus is known to have antibacterial and antifungal activity (Luiz et al., 2003), and its fruit bodies or mycelia have been suggested to have beneficial effects on nephritis, but studies on other physiological uses still remain insignificant.

With respect to conventional techniques, reference can be made to Korean Patent Number 10-1994-0025583 A that discloses Irpex lacteus polysaccharide KD102 having a therapeutic effect on nephritis, a preparation method therefor, and a pharmaceutical formulation containing same as an active ingredient and a preparation method therefor and to Korean Patent Number 2250261 A that discloses a composition for promoting growth of plants, the composition containing as an active ingredient a conversion product of lignin prepared through Irpex lacteus KACC 43133 fermentation. In addition, Korean Patent Number 1185823 discloses a transformant expressing two kinds of ligninases, a preparation method therefor, and a method for degrading endocrine disruptors by using same. However, the cited documents are different from the present disclosure in terms of purpose, configuration, and advantage.

### DISCLOSURE OF INVENTION

### Technical Problem

An aspect of the present disclosure is to provide an Irpex lacteus culture and a composition containing same as an active ingredient for prevention and treatment of diabetes mellitus. Another aspect of the present disclosure is to establish a mass culturing method for Irpex lacteus mycelia and to provide a composition containing an extract from the culture as an active ingredient for prevention and palliation of diabetes mellitus.

### Solution to Problem

To achieve the goals, the present disclosure provides a composition containing Irpex lacteus or a mycelial culture thereof for prevention or treatment of diabetes mellitus. In the present disclosure, various kinds of mushrooms were collected from nature and selection was made of mushrooms exhibiting antidiabetic functions. Of them, one mushroom species was found to have an excellent antidiabetic function and identified as Irpex lacteus before deposition. Additional experiments of the present inventors revealed that individual cultivars of Irpex lacteus produce similar secondary metabolites, exhibiting a consistent antidiabetic performance. Irpex lacteus can be obtained using a typical method. It may be granted from the Seed Bank in the Rural Development Administration National Institute of Agricultural Sciences or obtained by natural collection.

The present disclosure provides Irpex lacteus KACC 83046BP. In addition, the present disclosure provides a mycelial culture of Irpex lacteus KACC 83046BP. Also, the present disclosure provides optimal conditions of medium composition and culture environment for mass production of a mycelial culture of Irpex lacteus KACC 83046BP.

The mycelial culture may be obtained by culturing a mycelium derived from the Irpex lacteus KACC 83046BP strain.

The mycelial culture may be obtained by a method in which the Irpex lacteus KACC 83046BP strain is cultured at 18-27° C. for 5-7 days under aeration at 0.05-2 vvm while stirring at 25-100 rpm in a broth containing sucrose 0.2-2% (w/v), glucose 0.2-2% (w/v), starch 0.2-2% (w/v), soy flour 0.05-1.5% (w/v), yeast extract 0.05-1.5% (w/v), soy peptone 0.05-1.5% (w/v), MgSO₄ 0.001-0.005% (w/v), KH₂PO₄ 0.001-0.005% (w/v), K₂HPO₄ 0.001-0.005% (w/v), biotin (B7) 0.001-0.005% (w/v), and pyridoxine (B6) 0.001-0.005% (w/v), based on the total weight thereof and having a pH of 5-6.

The mycelial culture of Irpex lacteus KACC 83046BP obtained by the method contains beta glucan in an amount of 35.21% and extracellular polysaccharides in an amount of 44.34%, based on the dry weight thereof and can find advantageous applications in the industrial field.

The present disclosure provides a composition for prevention or treatment of diabetes mellitus wherein the composition contains the novel strain Irpex lacteus KACC 83046BP or a mycelial culture thereof. The composition for prevention or treatment of diabetes mellitus is characterized by at least one of the functions of reducing a blood bA1c level (%), a blood insulin level, a blood c-peptide level, a blood glucose level, and an OGTT level.

The present disclosure provides a method for mass production of Irpex lacteus KACC 83046BP mycelia or a culture thereof, the method comprising culturing the strain at 18-27° C. for 5-7 days under aeration at 0.05-2 vvm while stirring at 25-100 rpm in a broth containing sucrose 0.2-2% (w/v), glucose 0.2-2% (w/v), starch 0.2-2% (w/v), soy flour 0.05-1.5% (w/v), yeast extract 0.05-1.5% (w/v), soy peptone 0.05-1.5% (w/v), MgSO₄ 0.001-0.005% (w/v), KH₂PO₄ 0.001-0.005% (w/v), K₂HPO₄ 0.001-0.005% (w/v), biotin (B7) 0.001-0.005% (w/v), and pyridoxine (B6) 0.001-0.005% (w/v), based on the total weight thereof and having a pH of 5-6.

Components in the broth may have influences on the growth of the strain and the production of the desired product. The broth may contain as a carbon source at least one selected from the group consisting of maltose, glucose, lactose, starch, dextrin, sucrose, fructose, galactose, mannose, and oligosaccharides, but with no limitations thereto. Particularly, the broth contains sucrose, glucose, and starch, more particularly sucrose 0.2-2% (w/v), glucose 0.2-2% (w/v), and starch 0.2-2% (w/v), and far more particularly sucrose 0.5% (w/v), glucose 0.5% (w/v), and starch 0.5% (w/v).

The broth may contain as a nitrogen source at least one selected from the group consisting of soy flour, a yeast extract, L-glutamic acid, soy peptone, a malt extract, ammonium, calcium nitrate, potassium nitrate, and sodium nitrate, with no limitations thereto. The broth contains particularly soy flour 0.05-1.5% (w/v), a yeast extract 0.05-1.5% (w/v), and soy peptone 0.05-1.5% (w/v) and most particularly soy flour 0.1% (w/v), a yeast extract 0.1% (w/v), and soy peptone 0.1% (w/v).

The broth may contain at least one microelement selected from the group consisting of KH₂PO₄, ZnSO₄, MgSO₄, CuSO₄, FeSO₄, and CaCl₂), with no limitations thereto. The broth contains MgSO₄, KH₂PO₄, and MgSO₄, and more particularly MgSO₄ 0.001-0.005% (w/v), KH₂PO₄ 0.001-0.005% (w/v), and K₂HPO₄ 0.001-0.005% (w/v). Most particularly, the broth contains MgSO₄0.003% (w/v), KH₂PO₄ 0.001% (w/v), and K₂HPO₄ 0.002% (w/v).

The broth may contain a vitamin, and particularly vitamins B, C, and E, but with no limitations thereto. Particularly, the broth contains biotin (B7) 0.001-0.005% (w/v), and pyridoxine(B6) 0.001-0.005% (w/v), and more particularly biotin (B7) 0.003% (w/v) and pyridoxine(B6) 0.002% (w/v).

The broth may have a pH of 5-6 and particularly a pH of 5.5. The pH can affect the shape and activity of the protein by changing charges of the amine group or carboxyl group of amino acids, which are units of enzyme proteins important for cellular metabolism. In addition, a pH change in an external environment affects the ionization of nutrients for microbes and thus the uptake of nutrients by microbes. In addition, the broth with a pH less than 5 allows the aerobic, acidophiles algae or archaea to easily grow therein. On the other hand, basophiles actinomyces and mold fungi are apt to grow at a pH higher than 6. At such pH values, the mycelia do not grow.

The culturing may be conducted using a stirring bioreactor. The growth of the strain may vary depending on various factors including air supply, stirring speed, culture temperature, and culture time. It is thus important to establish an optimal culturing condition for the Irpex lacteus KACC 83046BP strain of the present disclosure.

The air supply may be conducted at an aeration rate of 0.02-1 vvm, particularly 0.1-0.5 vvm, and more particularly 0.2 vvm.

The stirring speed may be 25-100 rpm, particularly 50-100 rpm, and more particularly 60 rpm.

The culturing temperature may be 18-27° C. When the culturing temperature is below 18° C., the activity of the mycelia becomes dull, lengthening the period of culturing time, with the consequence of a decrease in productivity and a sudden rise in production cost. At a culturing temperature exceeding 27° C., the mycelia excessively grow and rapidly increase in mass, but with the production of the desired product only at an extremely low amount. A culturing temperature over 28° C. causes the mycelia to die. Most preferable is 23° C.

The culture duration is set to be 5 to 7 days and more particularly 6 days. When the culturing is conducted for less than 4 days, only a small amount of mycelia is obtained, along with a low content of the desired product. Even if the mycelia are cultured for more than 7 days, the content of the desired product does not increase any more, but rather gradually decreases. A culture duration exceeding 9 days causes the content of the desired product to drastically decrease.

Mycelia of Irpex lacteus KACC 83046BP or a culture thereof is obtained by culturing mycelia derived from the Irpex lacteus KACC 83046BP strain in broth. After culturing the strain, the culture containing the mycelia and the broth or the culture liquid obtained by removing the mycelial mass from the cell culture may be used. Preferred is the culture liquid obtained by removing the mycelial mass from the cell culture. The separated culture liquid may be concentrated in a non-heating manner in order to enhance a lyophilization process. Preferably, reverse osmosis (RO) may be employed.

The culture liquid may be prepared into powder by a typical drying method. Preferable is lyophilization. The culture may be the culture liquid obtained by culturing the strain in broth, or an extract obtained by adding a solvent to a powder dried from the culture liquid, the solvent being selected from the group consisting of water, a lower alcohol of C1-C4, acetone, n-hexane, dichloromethane, and ethyl acetate.

The lower alcohol of C1-C4 may be methanol, ethanol, propanol, isopropanol, butanol, etc. The extraction may be conducted by any one method selected from hot water extraction, cold precipitation, reflux condensation, solvent extraction, steam distillation, ultrasonic extraction, elution, and compression. In addition, a desired extract may be obtained by an additional typical fractionation process and may be further purified using a conventional purification method.

The mycelial culture of Irpex lacteus KACC 83046BP may be filtered through a filter press and the volume of the filtrate may be reduced by a concentration process. Reverse osmosis (RO) may be advantageous in terms of process rate and prevention of the heat-induced loss of the desired product. The concentrate may be used as a substance for a food or health functional food for prevention or alleviation of diabetes or may be added to other materials.

The concentrate may be powdered by lyophilization. The lyophilization may be conducted to a water content of 0.1% or less while maintaining a heating plate at 40° C. or less upon drying. A heating temperature of higher than 40° C. can reduce the drying time, but may cause thermal damages on protein and vitamin ingredients. Particularly, the lyophilization is carried out at 30° C. or less. The lyophilizate thus obtained is in a swollen state like cotton and can be finely pulverized using a mill. The powder may be used as a substance for a food or health function food for prevention and alleviation of diabetes or may be added to other materials.

Only functional ingredients may be separated and extracted from the lyophilizate. In this regard, the extraction may be conducted by any one method selected from hot water extraction, cold precipitation, reflux condensation, solvent extraction, steam distillation, ultrasonic extraction, elution, and compression. In addition, a desired extract may be obtained by an additional typical fractionation process and may be further purified using a conventional purification method.

The extract may include beta glucan, extracellular polysaccharides, and other functional substances.

The present disclosure provides a composition containing Irpex lacteus KACC 83046BP mycelia or a culture thereof as an active ingredient for prevention and treatment of diabetes mellitus.

With very high contents of β-glucan and extracellular polysaccharides, known to have a therapeutic effect on diabetes, the mycelial culture of Irpex lacteus KACC 83046BP exhibits excellent hyperglycemic effects.

The pharmaceutical composition may contain mycelia of Irpex lacteus KACC 83046BP or a culture thereof and a pharmaceutically acceptable excipient.

The mycelial culture of Irpex lacteus KACC 83046BP may be contained particularly in an amount of 0.001-50% by weight, more particularly in an amount of 0.001-40% by weight, and most particularly in an amount of 0.001-30% by weight, based on the total weight of the pharmaceutical composition.

The pharmaceutical composition may be formulated into oral dosage forms such as pulvis, granules, tablets, capsules, suspensions, emulsions, syrups, aerosols, etc. and parenteral dosage forms such as topical agents, suppositories, and sterile injections according to typical methods. The pharmaceutical composition may include a carrier, an excipient, and a diluent, as exemplified by lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil. The composition may be formulated with a commonly used diluent or excipient such as a filler, an extending agent, a binder, a humectant, a disintegrant, a surfactant, and the like. A solid preparation for oral administration includes a tablet, a pill, a pulvis, a granule, a capsule, and the like, and such a solid preparation is prepared by mixing a mycelial culture of Irpex lacteus KACC 83046BP according to the present disclosure with one or more excipients, for example, starch, calcium carbonate, sucrose, lactose, gelatin, and the like. In addition to the simple excipient, a lubricant such as magnesium stearate, talc, and the like may also be employed. A liquid preparation for oral administration includes a suspension, a liquid for internal use, an emulsion, a syrup, and the like. In this regard, a commonly used simple diluent, such as water and liquid paraffin, plus various excipients, such as a humectant, a sweetener, an aromatic, a preservative, and the like, may be included. A preparation for parenteral administration includes a sterile aqueous solution, a non-aqueous solvent, a suspension, an emulsion, a lyophilized preparation, and a suppository, and the like. Available as the non-aqueous solvent and the suspension are propylene glycol, polyethylene glycol, a vegetable oil, such as olive oil, an injectable ester, such as ethyl oleate, and the like. As a suppository base, Witepsol, Macrogol, Tween 61, cacao butter, laurin fat, glycerol, gelatin, and the like may be used.

The dose of the pharmaceutical composition according to the present disclosure may vary depending on various factors including the age, sex and body weight of a subject to be treated, a particular disease or pathological condition to be treated, severity of disease or pathological condition, administration route and the judgment of a prescriber. Determination of the effective dose may be made by those skilled in the art on the basis of the above-mentioned factors. In general, the effective dose may be in the range of 1000-5000 mg/day and particularly in the range of 1000-3000 mg/day. The administration may be performed once a day or in multiple doses. The dosage in no way limits the scope of the prevent disclosure.

The pharmaceutical composition of the present disclosure may be administered to mammals such as rats, livestock, humans, etc. via various routes. All the modes of administered may be contemplated. By way of example, administration may be conducted orally, rectally, or intravenous, intraperitoneal, intramuscular, subcutaneous, intracervical, or intra-cerebrovascular injection. In addition, the pharmaceutical composition of the present disclosure, which is derived from the natural product, is almost free of toxicity and side effects and as such, can be safely used even when taken for a long period of time for the purpose of prevention.

In addition, the present disclosure provides a health function food including Irpex lacteus KACC 83046BP mycelia or a culture thereof for prevention and alleviation of diabetes mellitus.

The health functional food may contain a sitologically acceptable supplemental additive in addition to the mycelial culture of Irpex lacteus KACC 83046BP.

The mycelial culture of Irpex lacteus KACC 83046BP may be used particularly in an amount of 0.001-90% by weight, more particularly in an amount of 0.001-70% by weight, and most particularly in an amount of 0.001-50% by weight, based on the total weight of the health functional food.

The intake of the mycelial culture of Irpex lacteus KACC 83046BP in the health function food may be 2,000 mg or less per dosage and 5,000 mg or less a day. Most particularly, the mycelial culture is taken at a unit dose of 1,000 mg three to four times a day.

The health functional food of the present disclosure may be in the form of a tablet, a capsule, a pill, or a liquid. The culture of the present disclosure may be added to various foods including beverages, gum, tea, vitamin complexes, health functional foods, and the like.

### Advantageous Effects of Invention

The present disclosure pertains to a mycelial culture of Irpex lacteus and a composition containing same as an active ingredient for prevention and treatment of diabetes mellitus. The mycelial culture of Irpex lacteus is expected to find advantageous applications in preventing and alleviating diabetes mellitus as it was found to have high contents of extracellular polysaccharides and beta-glucan which are prophylactically and therapeutically effective for diabetes mellitus and safe as a natural substances and to exhibit an excellent anti-diabetic effect.

### BRIEF DESCRIPTION OF DRAWINGS

FIG. 1 is ITS1 sequence of the novel strain Irpex lacteus KACC 83046BP according to the present disclosure.

FIG. 2 is ITS4 sequence of the novel strain Irpex lacteus KACC 83046BP according to the present disclosure.

FIG. 3 is a phylogenetic tree showing a taxonomical position of the Irpex lacteus KACC 83046BP strain.

FIG. 4 is a copy of the receipt issued for deposit of the Irpex lacteus KACC 83046BP strain.

### BEST MODE FOR CARRYING OUT THE INVENTION

Mushrooms are rich in carbohydrates, proteins, lipids, minerals, vitamins, etc., and contain a large amount of biologically active substances including beta-glucan and extracellular polysaccharides. In addition, it is scientifically proven in numerous studies that certain mushrooms have excellent functions in the prevention and treatment of diseases. However, only a small portion of mushrooms have been used so far, and there are countless mushrooms that need to be studied in the natural world. Mushrooms that have not been studied so far are also highly likely to be effective in disease prevention and treatment, so research is needed to expand the utilization of natural resources by identifying the safety and functionality of unused mushrooms.

Leading to the present disclosure, intensive and thorough research conducted by the present disclosure resulted in the finding that Irpex lacteus is abundant in beta-glucan and extracellular polysaccharides and have anti-diabetic activity.

Hereinafter, preferable embodiments of the present disclosure will be described in detail. However, the present disclosure is not limited to the embodiments described herein, and can be embodied in many different forms. Rather, these embodiments are provided so that this disclosure will be thorough and complete and will fully convey the scope of the disclosure to those skilled in the art. Unless otherwise defined, all technical and scientific terms used herein have the same meaning as commonly understood by one of ordinary skill in the art to which this presently described subject matter belongs.

### EXAMPLE 1. Collection and Identification of Irpex lacteus KACC 83046BP

1.1. Collection of Irpex lacteus KACC 83046BP

Irpex lacteus can be obtained using a typical collection method for microbial strains. The strain can be granted from the Seed Bank in the Rural Development Administration National Institute of Agricultural Sciences or obtained by natural collection. One of the strains collected by the present inventors from nature was found to have excellent anti-diabetic activity and deposited in the name of Irpex lacteus KACC 83046BP.

Irpex lacteus which grows wild at oak habitats in Mungyeong Saejae Jooheul Mountain (San 40, Sangcho-ri, Mungyeong-eup, Mungyeong-si, Kyengsanbuk-do) was collected down to the base thereof. To remove germs, alcohol (75%) was sprayed over the surface of the fruiting body on a clean bench. After being left for 5 minutes, the fruiting body was cut in half and the tissue inside the base was cut out to a size of 1-2 mm, followed by passage in a plate (100 mm) containing potato dextrose agar (PDA, Difco). The PDA medium was supplemented with ampicillin (200 mg/L) to prevent bacterial contamination during cultivation. The agar plate was incubated at 24° C. for 7 days in an incubator. The mycelia grown with the hyphae rampant in the plate was cut using a 5-mm cork borer and passaged gain on a fresh PDA medium. The re-passaged strain was incubated at 24° C. for 5 days in an incubator to obtain a parent strain.

1.2. Identification of Irpex lacteus KACC 83046BP

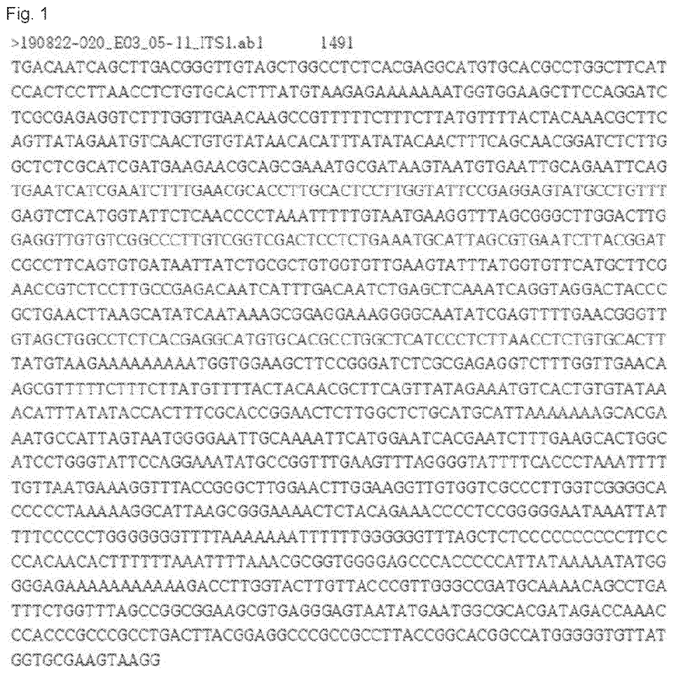

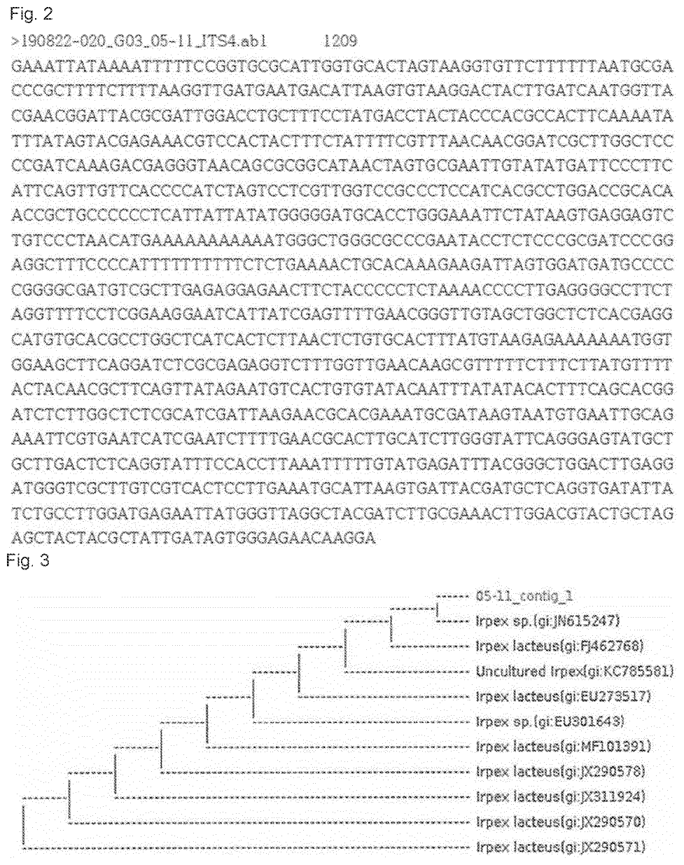

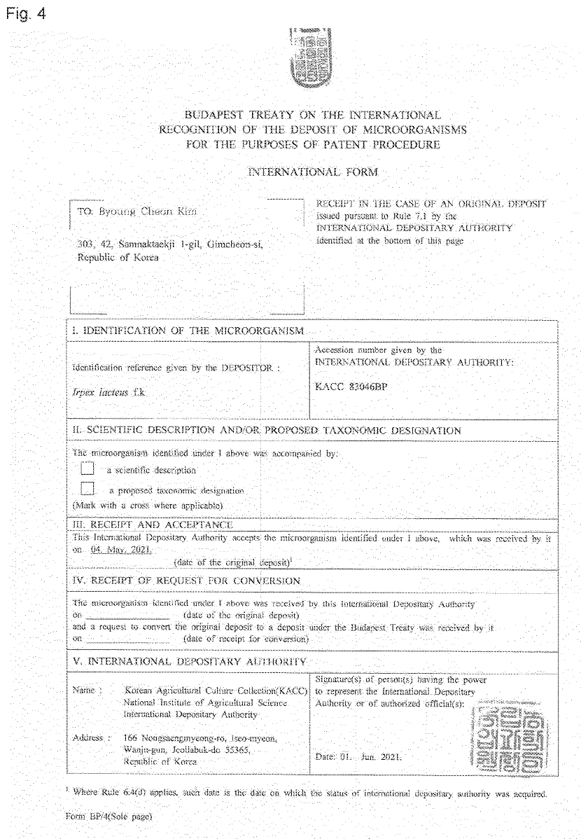

The parent strain obtained by collection and cultivation in Example 1.1 was subjected to ITS sequencing in Macrogen Inc. (2019, Korea) and identified to be Irpex lacteus. FIGS. 1 and 2 show ITS1 (SEQ ID NO: 1) and ITS4 (SEQ ID NO: 2) sequences of Irpex lacteus, respectively and FIG. 3 is a phylogenetic tree for the Irpex lacteus KACC 83046BP strain according to the present disclosure. For patent application, the isolated and identified strain was deposited with the international depositary authority Korean Agricultural Culture Collection National Institute of Agricultural Science on Jun. 1, 2021 (FIG. 4).

### EXAMPLE 2. Establishment of Optimal Culturing Condition for Irpex lacteus KACC 83046BP

2.1 Composition of Nutrient Medium for Culturing Irpex lacteus KACC 83046BP

In this test, a 100-1 bioreactor installed in the Marine Industry Research Institute for East Sea Rim was used. All experiments were conducted in the following conditions. Broths different in composition were autoclaved at 123° C. for 30 minutes under 1.3 bar and cooled to 23° C. before inoculation of 600 ml of the Irpex lacteus strain thereinto. Culturing was carried out at 23±1° C. and a pH of 5.5 while air was fed at a rate 0.1 vvm.

1) Effect of Carbon Source on Mycelial Growth of Irpex lacteus

Examination was made of the effect of carbon sources on the mycelial growth of Irpex lacteus and the results are summarized in Table 1, below. As shown in Table 1, sucrose was the best carbon source in terms of the weight of mycelia and glucose was the next best.

TABLE 1

| Broth | 80 l | | | | | |
|---|---|---|---|---|---|---|
| % Carbon source | 1.5% (W/V) | | | | | |
| Type of carbon source | Sucrose | Glucose | Starch | Fructose | Lactose | Maltose |
| Weight of mycelium (g/100 ml) | 21.22 g | 22.25 g | 19.13 g | 15.48 g | 14.23 g | 16.31 g |

2) Effect of Nitrogen Source on Mycelial Growth of Irpex lacteus

Examination was made of the effect of nitrogen sources on the mycelial growth of Irpex lacteus and the results are summarized in Table 2, below. As shown in Table 2, a yeast extract was the best carbon source in terms of the weight of mycelia and soy peptone was the next best.

TABLE 2

| Broth | 80 l | | | | | |
|---|---|---|---|---|---|---|
| Carbon source | Glucose 1.5%(W/V) | | | | | |
| % Nitrogen source | (W/V) | | | | | |
| Type of nitrogen | Soy | Soy | Yeast | Ammonium | Calcium | Potassium |
| source | flour | peptone | extract | | nitrate | nitrate |
| Mycelial mass | 35.41 g | 37.45 g | 44.91 g | 14.33 g | 17.52 g | 14.21 g |
| (g/100 ml) | | | | | | |

3) Effect of Mineral on Mycelial Growth of Irpex lacteus

Examination was made of the effect of minerals on the mycelial growth of Irpex lacteus and the results are summarized in Table 3, below. As shown in Table 3, K₂HPO₄ was the best carbon source in terms of the weight of mycelia and MgSO₄ was the next best.

TABLE 3

| Culture liquid | 80 l | | | | | |
|---|---|---|---|---|---|---|
| Carbon source | Glucose 1.5% (W/V) | | | | | |
| Nitrogen source | Yeast extract 0.2%(W/V) | | | | | |
| % Mineral | 0.001%(W/V) | | | | | |
| Type of mineral | KH₂PO₄ | MgSO₄ | K₂HPO₄ | FeSO₄ | ZnSO₄ | CuSO₄ |
| Mycelial mass (g/100 ml) | 43.22 g | 44.32 g | 45.11 g | 23.12 g | 19.30 g | 15.05 g |

4) Effect of Vitamin on Mycelial Growth of Irpex lacteus

Examination was made of the effect of vitamins on the mycelial growth of Irpex lacteus and the results are summarized in Table 4, below. As shown in Table 4, biotin was the best carbon source in terms of the weight of mycelia and pyridoxine was the next best.

TABLE 4

| Culture liquid | 80 l | | | |
|---|---|---|---|---|
| Carbon source | Glucose 1.5% (W/V) | | | |
| Nitrogen source | Yeast extract 0.2% (W/V) | | | |
| Mineral | MgSO₄ 0.001, KH₂PO₄ 0.001%, K₂HPO₄ 0.001% | | | |
| % Vitamin | 0.005% (W/V) | | | |
| Vitamin type | Thiamine | Pyridoxine | Biotin | Ascorbic acid |
| Mycelial mass | 22.34 g | 28.83 g | 29.72 g | 19.32 g |
| (g/100 ml) | | | | |

From the above experiments for effects of nutrients on mycelial growth of Irpex lacteus, ratios of the most suitable nutrients are as follows: carbon source: sucrose 0.5% (w/v), glucose 0.5% (w/v), and starch 0.5% (w/v); nitrogen source: soy flour 0.1% (w/v), yeast extract 0.1% (w/v), and soy peptone 0.1% (w/v); mineral: MgSO₄ 0.003% (w/v), KH₂PO₄ 0.001% (w/v), and K₂HPO₄ 0.002% (w/v); and vitamin: biotin (B7) 0.003% (w/v) and pyridoxine (B6) 0.002% (w/v).

2.2. Establishment of Environmental Condition for Culturing Irpex lacteus KACC 83046BP

Examination was made of the effect of environmental conditions on the mycelial growth of Irpex lacteus. In this regard, a broth was prepared at the ratio of the most suitable nutrients identified in Example 2.1. and 600 ml of the Irpex lacteus strain was inoculated thereinto and cultured for 9 days.

### 1) Stirring Speed

Examination was made of the effect of stirring speeds on the mycelial growth of Irpex lacteus and the results are summarized in Table 5, below. As shown, the most suitable stirring speed was 60 rpm.

TABLE 5

| Stirring speed (rpm) | Mycelial mass (g/l) |
|---|---|
| 25 | 5.4 |
| 50 | 6.9 |
| 60 | 8.8 |
| 70 | 8.2 |
| 80 | 6.3 |
| 100 | 5.4 |

### 2) Aeration

Examination was made of the effect of aeration rates on the mycelial growth of Irpex lacteus and the results are summarized in Table 6, below. As shown, the most suitable aeration rate was 0.2 vvm.

TABLE 6

| Aeration (vvm) | Mycelial mass (g/l) |
|---|---|
| 0.05 | 7.8 |
| 0.1 | 8.6 |
| 0.2 | 8.8 |
| 0.5 | 8.1 |
| 1 | 7.7 |
| 2 | 7.4 |

### 3) Culturing Temperature

Examination was made of the effect of culturing temperatures on the mycelial growth of Irpex lacteus and the results are summarized in Table 7, below. As shown, the most suitable culturing temperature was 23° C.

TABLE 7

| Culturing Temp. (° C.) | Mycelial mass (g/l) |
|---|---|
| 18 | 6.3 |
| 19 | 6.8 |
| 20 | 7.6 |
| 22 | 8.5 |
| 23 | 8.8 |
| 24 | 8.6 |
| 25 | 8.1 |
| 26 | 4.9 |
| 27 | 0.3 |

4) pH

Examination was made of the effect of pH on the mycelial growth of Irpex lacteus and the results are summarized in Table 8, below. As shown, the most suitable pH was 5.5.

TABLE 8

| pH | Mycelial mass (g/l) |
|---|---|
| 4.0 | 3.5 |
| 4.5 | 6.2 |
| 5.0 | 8.6 |
| 5.5 | 8.8 |
| 6.0 | 6.4 |
| 6.5 | 4.9 |
| 7.0 | 2.2 |

### 5) Culturing Duration

Examination was made of the effect of culturing duration on the mycelial growth of Irpex lacteus and the results are summarized in Table 9, below. As shown, the most suitable culturing duration was 7 days.

TABLE 9

| Culturing period (day) | Mycelial mass (g/l) |
|---|---|
| 3 | 3.7 |
| 4 | 6.1 |
| 5 | 8.6 |
| 6 | 8.7 |
| 7 | 8.8 |
| 8 | 7.9 |
| 9 | 7.2 |

From the above experiments or effects of the environmental conditions on mycelial growth of Irpex lacteus, the most suitable environmental conditions were set to be 60 rpm for stirring speed, 0.2 vvm for aeration rate, 23° C. for culturing temperature, 5.5 for pH, and 7 days for culturing time.

### EXAMPLE 3. Preparation of Concentrate, Extract, and Lyophilizate of Culture of Irpex lacteus KACC 83046BP

### 3.1. Preparation of Concentrate by Reverse Osmosis (RO)

The mycelial culture is too bulky, so there are many restrictions on storage and transportation. In order to solve this problem, it is necessary to reduce the volume. In general, hydrothermal pressure reduction and reverse osmosis methods are used to reduce the volume of a liquid. Reverse osmosis can remove up to 90% (12 times) of pure water without heating, but the culture was concentrated 6-fold in consideration of material characteristics and economic feasibility.

### 3.2. Extraction of Functional Ingredient by Chelating

A chelating method was used to reduce the volume of the mycelial culture by more than 13 times. The chelating method is a technique using a precipitant to extract only functional ingredients. As a precipitant, aluminum chloride, iron oxide, iron sulfate, calcium chloride, cyclodextrin, etc. may be used. The amount of the precipitant is preferably 0.2-2% of the culture volume, with the most preference for 0.5%. After addition of a precipitant, the culture was induced to precipitate for 1-2 hours at 20-25° C. while being stirred at 30 rpm. Completion of the precipitation was followed by centrifugation at 10,000-15,000 rpm. Most suitable was 12,000 rpm.

After centrifugation, the pellet thus formed was recovered. Functional ingredients in the pellet were extracted using a buffer. The buffer was prepared by mixing NaCl, KCl, Na2PO₄, and KH₂PO₄ in distilled water. The buffer was prepared at a desired concentration ratio from 1 to 200 times. The precipitate and the buffer were mixed at 30 rpm for 30-240 min in a mixer, followed by centrifugation (12,000 rpm) to give an extract as a supernatant.

### 3.3. Preparation of Lyophilizate

The concentrate and lyophilizate respectively obtained in Examples 3.1. and 3.2. were prepared into powder by lyophilization. Lyophilization may be conducted by a typical method using a freeze drier. The process was carried out to a water content of 3% or less, with the temperature set to be −40° C. for freezing and 30° C. for vaporization. The lyophilizate was obtained in an amount of 1.5-1.8% of the weight of the culture. Since the lyophilizate was in a swollen state like cotton, it was powdered using a grinder.

### EXAMPLE 4. Analysis of Functional Ingredient of Irpex lacteus KACC 83046BP

The lyophilizate of the mycelial culture of Irpex lacteus was quantitatively assayed for index ingredients and the results are summarized in Table 10, below.

TABLE 10

| Index material | Content |
|---|---|
| β-Glucan | 35.21(%/g) |
| Extracellular polysaccharide (EPS) | 44.34(%/g) |

The lyophilizate of the mycelial culture of Irpex lacteus was measured to have the beta-glucan content twice or more times than 16.4% in Phellinus linteus and 15% in Ganoderma lucidum Karsten. In addition, extracellular polysaccharides were found at a very high content in the lyophilizate, compared to 1.25% in Chaga mushroom, 6.0% in Phellinus linteus, and 1.3% in Ganoderma lucidum Karsten. Beta-glucan and extracellular polysaccharides are known as anti-diabetic ingredients of mushrooms. Accordingly, it could be estimated that Irpex lacteus mushroom had a very good antidiabetic effect than other mushrooms.

### EXAMPLE 5. Assay for Antidiabetic Effect of Irpex lacteus KACC 83046BP

Diabetes mellitus is a type of metabolic diseases caused by insufficient insulin secretion, insulin dysfunction, etc., and is characterized by hyperglycemia accounting for a high blood glucose level. Hyperglycemia provokes various symptoms and signs and results in secretion of glycose in the urine.

Diabetes mellitus is divided into type 1 and type 2. Type 1 diabetes, previously called juvenile diabetes, is a disease caused by the inability to produce insulin at all. Type 2 diabetes in which cells respond poor to insulin is characterized by insulin resistance (inability of cells to effectively burn glucose due to a decrease in insulin performance of lowering blood glucose levels). In type 2 diabetes, environmental factors such as a high calorie, high fat, and high protein diet according to westernization of diet, lack of exercise, and stress seem to play a major role. Type 2 diabetes can also occur due to defects in certain genes, pancreatic surgery, infection, and drugs.

In mild hyperglycemia, most patients do not feel symptoms or the symptoms are vague so that it is difficult for the patients to think of diabetes. If the blood sugar in a person rises a lot, he or she gets thirsty and drinks a lot of water, and the amount of urine will increase, thus making him or her go to the bathroom more often. In addition, weight loss occurs. If hyperglycemia is maintained for a long time, various complications occur in the body, typical of which are retinopathy, kidney failure, neuropathy, and the increased risk of cardiovascular disease.

Currently available hypoglycemic agents are largely divided into oral medications and injections. There are two main oral hypoglycemic agents including insulin secretagogues and insulin sensitizers. Examples of the insulin secretagogues include sulfonylureas and meglitinides.

When administered alone, insulin sensitizers do not cause hypoglycemia at all. Insulin sensitizers can be exemplified by biguanides and thiazolidinediones. Also, acarbose and voglibose, which act to delay absorption of carbohydrates in the small intestine, are used as hyperglycemic agents.

A GLP-1 agonist has been developed using the action of the hormone glucagon-like peptide-1 (GLP-1) that lowers blood sugar levels. In addition, there are DPP-4 inhibitors and SGLT2 inhibitors that inhibit the activity of the enzyme dipeptidyl peptidase-4 (DPP-4) rapidly inactivating GLP-1, and the renal reabsorption of glucose, respectively.

In principle, injectable insulin is administered by subcutaneous injection, and the method of administration differs depending on the time of action. Insulin injections show a faster blood sugar lowering effect than oral drugs, and can be used safely even in environments where oral drugs cannot be used, and there is no dose limitation. However, insulin injections are disadvantageous in terms of rejection of needles and difficulty in administration (the Medical Information of Seoul National University Hospital).

As type 2 diabetic models, db/db or ob/ob mice are usually used. The db/db mice are used to model diabetes due to the mutation on the leptin receptor gene. The mice are established as insulin-non-dependent diabetes models that suffer from diabetes due to the lack of signaling performance with the deficiency of leptin receptors (Park 15 IS (2004): The efficacy test guideline for health functional food (I), Korea Food & Drug Administration, 179-215).

In this experiment, the test material lyophilizate of a mycelial culture of Irpex lacteus KACC 83046BP was orally administered to the db/db mice, which are an animal model of diabetes, for three weeks and examined for hypoglycemic effect. This test was conducted in the Local Food Institute of Gimcheon University.

### 5.1 Experimental Preparation

### 1) Preparation of Materials and Test Substances

For use in tests, sterile water for injection (excipient) was used as a negative control while Diabex tablet (metformin phosphate, 500 mg) commercially available from Dai Han Pharm. Co., Ltd. (Kyeonggi-do) was used as a positive control. A test substance was divided into three groups: high-, medium-, and low-dose groups, which were prepared in suspension by adding the same amount of the test substance to different amounts of the sterile water for injection.

### 2) Test System and Rearing Environment

TABLE 11

| | |
|---|---|
| Species and Lineage | db/db mice, 57BLKS/J-db/db |
| Provider | Central Lab. Animal Inc./Seoul |
| No. of animal | Female, 50 mice |
| Weeks after birth | 7 |
| Body weight upon administration | 30.32-35.14 g |

This experiment was carried out in a separate animal facility area of a clinical laboratory. During the experiment, db/db mice were reared at a temperature of 23±1° C. and a relative moisture of 55±5% under light for 12 hours at an intensity of 200 lux in a natural ventilation state. For use as a feed for the db/db mice, Product Data-D10001 (Research diets, USA) that had been identified to have no influences on the experimental data was purchased from Central Lab. Animal Inc. The animals were allowed to freely access water that was purified from tap water through reverse osmosis. The purchased experimental animals were acclimated for 6 days before the experiment and observed for aberrant symptoms once a day.

### 3) Division of Test Group and Administration of Test Substance

To classify the test group, the animals were ranked by measuring the weight thereof, and then randomly distributed and grouped as shown in Table 12 so that the average blood glucose and body weight among the groups were similar.

TABLE 12

| | | | | Administration | | | |
|---|---|---|---|---|---|---|---|
| | | No. of | Animal | Standard | Dose | Administered | |
| Group | Sex | animal | No. | (mL/kg/day) | (mg/kg) | substance | Route |
| G1 | Male | 10 | 1-10 | 10 | 0 | Excipient | Oral |
| G2 | Male | 10 | 11-20 | 10 | 500 | Irpex lacteus | |
| G3 | Male | 10 | 21-30 | 10 | 750 | Irpex lacteus | |
| G4 | Male | 10 | 31-40 | 10 | 1,000 | Irpex lacteus | |
| G5 | Male | 10 | 41-50 | 10 | 400 | Diabex 500 mg | |

* G1: excipient control, G2-G4: test substance administered, G5: control administered. The dosages were set to be 1000 mg/kg/day four times as much as the clinical dose (250 mg/kg) as the upper limit, and 750 mg/kg/day, and 500 mg/kg/day as lower values. Administration was conducted as indicated in Table 13, below.

TABLE 13

| | |
|---|---|
| No. of Administration | Orally administered once a day for |
| & Duration | three weeks, 21 times in total |
| Administration | A dose of 10 mL/kg based on weight |
| Standard | measured on the day of administration |
| Administration | Experimental animals fixed and forcibly administered orally |
| Modality | using the oral administration aids metal sonde and syringe |

### 5.2 Experiment Practice

### 1) Observation of General Symptoms and Measures

The first day of administration of the test substance was set as Day 1. During the feeding and observation period, the status of death, observation was made of the type of general symptoms, the date of onset, and the severity of symptoms once a day and the results were recorded for each individual. Individuals with worsening general symptoms were isolated and excluded from evaluation of test results.

### 2) Monitoring of Blood Glucose Level

Blood glucose levels were measured once a week for three weeks during the experiment. In order to objectively measure blood glucose levels, all experimental animals were blocked from eating feed 6 hours before blood glucose measurement on the day of blood glucose measurement. Blood glucose levels were measured using a portable blood glucose meter (Accu-Check Performa/USA).

### 3) Oral Glucose Tolerance Test (OGTT)

(1) The OGTT measures the body's ability to use glucose.

(2) After 6 days of acclimatization, OGTT test animals were fasted for 16 hours before OGTT (water provided).

(3) Blood glucose was measured 30 minutes before administration of the test substance and once again 30 minutes later, that is, just before glucose administration.

(4) At 30, 60, 90, 120, and 240 min after administration of oral glucose at a dose of 2 g/10 mL/kg, blood was taken from tail veins and measured for glucose levels using a blood glucose meter (Accu-Check Performa).

### 4) Autopsy and Histopathological Examination

All the experimental animals were anesthetized by inhalation with CO2 and autopsied by laparotomy, and blood was collected from the posterior vena cava. After blood collection, the animals were killed by excision of the abdominal aorta and posterior vena cava and exsanguination. For histopathological examination, hematoxylin & eosin (H&E) staining was conducted to measure numbers and diameters of pancreatic islets and proportions of zymogen granules per unit area of exocrine pancreatic portions.

### 5) Clinical Pathology

### Blood Collection

Of the blood collected for clinical pathology on the day of the end of the experiment, about 200 μL was used for analyzing glycated hemoglobin (HbA1c) while the remainder was left for 30 min or longer at room temperature in a vacutainer tube containing a clot activator and then centrifuged at 3000 rpm for 10 min. The serum obtained by centrifugation was transferred to a new tube and subjected to hemato-biochemical examination.

### Hemato-Biochemical Examination

Using a hemato-biochemical analyzer, whole blood and sera which had been stored separately were subjected to hemato-biological examination for total cholesterol (TCHO), aspartate aminotransferase (AST), high-density lipoprotein (HDL), low-density lipoprotein (LDL), alanine aminotransferase (ALT), and glycated hemoglobin (HbA1c).

Measurement of Blood Insulin and c-Peptide

C-peptide is a substance that is secreted when proinsulin, a precursor of insulin, is degraded and released. The peptide, although lacking bioactivity, accounts for the secretion state of insulin. In addition, with a long half-life, C-peptide is used as an index to evaluate insulin secretion. The sera stored in separation were quantitatively assayed for insulin and c-peptide, using an ELISA kit (Shibayagi, AKRIN-011T and AKRCP-031).

### Statistics

Statistical analysis of measurements was conducted using the IBM SPSS package (SPSS Statistics 22 for Medical Science). Comparison was made among the excipient control, the test substance-administered group, and the control-administered group, with statistical significance at p<0.05.

### 5.3 Test Results

As a result of observation of general symptoms during the experiment period, no observation was made of abnormal symptoms or death animals due to the test substance. Weight measurements indicated a significant change in none of the experimental groups. This was true of feed intake.

1) Blood glucose level after repeated administration for three weeks

As a result of measurement for three weeks, the test substance-administered groups G3 (700 mg/kg/day) and G4 (1000 mg/kg/day) significantly decreased in blood glucose level (P<0.05), compared to the negative control. The blood glucose level in the positive control exhibited a significant decrease (P<0.05) during the experimental period, compared to the negative control and the vehicle control. The results are summarized in Table 14, below.

TABLE 14
Blood glucose level (mg/dL)

| Group | Day 1 | Week 1 | Week 2 | Week 3 |
|---|---|---|---|---|
| G1 | 361 ± 11 | 459 ± 35 | 551 ± 44 | 527 ± 22 |
| G2 | 362 ± 24 | 451 ± 13 | 533 ± 22 | 495 ± 24 |
| G3 | 359 ± 12 | 433 ± 23 | 478 ± 23* | 463 ± 22* |
| G4 | 366 ± 33 | 428 ± 41 | 463 ± 21* | 430 ± 35* |
| G5 | 365 ± 24 | 371 ± 12* | 452 ± 13* | 436 ± 11* |

*significantly different from G1, P < 0.05

2) Oral glucose tolerance test (OGTT) result

As a result of OGTT measurement, the test substance-administered groups G2 (500 mg/kg) and G3 (750 mg/kg) showed a tendency to decrease in blood sugar, compared to the negative control group G1, but with no significance. However, the test substance-administered group G4 (1000 mg/kg) decreased in blood glucose from 120 min after glucose administration, compared to the negative control G1, with statistical significance (P<0.05). From 30 min after administration, a significant decrease in blood glucose was steadily observed in the positive control (G5), compared to the negative control (G1) (P<0.05 or P<0.01). The results are summarized in Table 15, below.

TABLE 15
Blood glucose level (mg/dL)

| Group | −30 min | 0 min | 30 min | 60 min | 90 min | 120 min | 240 min |
|---|---|---|---|---|---|---|---|
| G1 | 361 ± 11 | 492 ± 31 | 675 ± 21 | 641 ± 21 | 624 ± 14 | 562 ± 35 | 402 ± 21 |
| G2 | 362 ± 34 | 564 ± 22 | 669 ± 22 | 636 ± 31 | 588 ± 12 | 459 ± 45 | 382 ± 52 |
| G3 | 354 ± 13 | 523 ± 20 | 658 ± 11 | 598 ± 22 | 542 ± 22 | 421 ± 13 | 335 ± 32 |
| G4 | 366 ± 31 | 504 ± 14 | 642 ± 34 | 584 ± 43 | 513 ± 33 | 384 ± 22* | 286 ± 21* |
| G5 | 265 ± 11 | 385 ± 33 | 421 ± 29* | 343 ± 12** | 246 ± 26** | 208 ± 11** | 174 ± 22** |

*significantly different from G1, P < 0.05
**significantly different from G1, P < 0.01

3) Histopathological examination result

As a result of histopathological examination, no significant changes in the number and diameter (μm) of islets (/cm²) and zymogen granules %/mm² of exocrine were observed in the test substance-administered groups G2 (500 mg/kg) and G3 (750 mg/kg), compared to the negative control G1. However, the test substance-administered group G4 (1000 mg/kg) was observed to decrease in the number and diameter (m) of islets (/cm²) and increase in zymogen granules %/mm² of exocrine, with significance (P<0.01). Observation was made of a decrease in the number and diameter (m) of islets (/cm²) and an increase in zymogen granules %/mm² of exocrine for the positive control (G5), compared to the negative control (G1), with significance (P<0.05 or P<0.01). The results are summarized in Table 16, below.

TABLE 16
Histopathological examination

| | | | Zymogen granules |
|---|---|---|---|
| | Pancreatic islet | | (%/mm² of |
| | No. of pancreatic | Diameter of | exocrine |
| Group | Islets (islets/cm²) | islet (μm) | pancreas) |
| G1 | 15.11 ± 0.8 | 165.43 ± 4.8 | 25.66 ± 0.8 |
| G2 | 14.32 ± 0.6 | 162.44 ± 5.1 | 27.86 ± 0.6 |
| G3 | 12.45 ± 0.5 | 158.23 ± 4.5 | 31.22 ± 0.8 |
| G4 | 5.71 ± 0.2** | 115.22 ± 4.6** | 35.25 ± 0.5** |
| G5 | 6.25 ± 0.1** | 126.25 ± 5.3* | 41.39 ± 0.7** |

*significantly different from G1, P < 0.05
**significantly different from G1, P < 0.01

4) Hemato-biochemical examination result

As a result of hemato-biochemical examination, no significant changes in HbA1c (%), TCHO, LDL, and HDL were observed in the test substance-administered groups G2 and G3, compared to the negative control G1. However, a significant decrease of AST (P<0.01) was observed, along with a tendency to decrease in ALT. The test substance-administered group G4 was observed to significantly decrease in HbA1c (%) (P<0.05) and also in HDL, AST, and ALT (P<0.01), compared to the negative control. The positive control G5 was observed to decrease in HbA1c (%), TCHO, and HDL, compared to the negative control, with significance (P<0.01 or P<0.05), but not significantly differ in terms of LDL, AST, and ALT. The results are summarized in Table 17, below.

TABLE 17
Hemato-Biochemical Examination

| | | TCHO | HDL | LDL | AST | ALT |
|---|---|---|---|---|---|---|
| Group | HbA1c(%) | (mg/dL) | (mg/dL) | (mg/dL) | (U/L) | (U/L) |
| G1 | 7.7 ± 0.4 | 134.9 ± 1.3 | 105.7 ± 3.3 | 28.1 ± 2.2 | 119.6 ± 4.3 | 73.4 ± 5.7 |
| G2 | 7.2 ± 0.1 | 132.1 ± 8.4 | 105.4 ± 5.4 | 28.0 ± 4.4 | 88.7 ± 6.2** | 59.6 ± 3.2 |
| G3 | 7.1 ± 0.3 | 129.4 ± 2.1 | 102.2 ± 2.1 | 27.4 ± 3.0 | 84.8 ± 3.7** | 54.8 ± 3.6 |
| G4 | 6.6 ± 0.3* | 121.3 ± 2.6 | 89.1 ± 3.5** | 25.6 ± 2.5 | 80.3 ± 4.4** | 50.5 ± 2.9** |
| G5 | 6.8 ± 0.2* | 116.7 ± 3.2* | 88.2 ± 4.2** | 25.9 ± 2.7 | 116.5 ± 4.6 | 72.1 ± 4.5 |

*significantly different from G1, P < 0.05
**significantly different from G1, P < 0.01

5) Blood Insulin and c-Peptide Measurement Result

As a result of blood insulin measurement, observation was made of a decrease tendency in the test substance-administered group G3 (750 mg/kg) and a significant decrease in the test substance-administered group G4 (1000 mg/kg), compared to the negative control (G1). A significant decrease was also observed in the positive control (G5), compared to the negative control (G1). The results are summarized in Table 18.

TABLE 18

| Group | Blood insulin level (ng/ml) |
|---|---|
| G1 | 3.14 ± 0.44 |
| G2 | 3.11 ± 0.42 |
| G3 | 2.50 ± 0.38 |
| G4 | 2.12 ± 0.31 |
| G5 | 1.96 ± 0.39 |

As a result of blood c-peptide measurement, a significant decrease was observed in the test substance-administered groups G3 (750 mg/kg) and G4 (1000 mg/kg), compared to the negative control (G1), with significance (P<0.01 or P<0.05). A significant reduction was observed in the positive control (G5), compared to the negative control (G1) (P<0.01). The results are summarized in Table 19.

TABLE 19

| Group | Blood C-peptide level (pg/mL) |
|---|---|
| G1 | 994.5 ± 93.6 |
| G2 | 885.1 ± 77.4 |
| G3 | 707.3 ± 74.5* |
| G4 | 612.4 ± 76.2** |
| G5 | 459.7 ± 82.3** |

*significantly different from G1, P < 0.05
**significantly different from G1, P < 0.01

### EXAMPLE 5.4. Discussion and Conclusion

The experiments were performed to evaluate the antidiabetic effect of a culture of Irpex lacteus (accession No. KACC 83046BP) after repeated oral administration to db/db mice for three weeks.

As seen in the foregoing, the results of the three-week repeated administration test indicate that the culture significantly decreased blood glucose levels in the test substance-administered groups G3 and G4 from week 2 and exhibited a higher decrease effect than the positive control substance in week 3, suggesting a more effective decrease in blood sugar level over a longer administration period

In the oral glucose tolerance test, the test substance-administered group G4 (1000 mg/kg) exhibited a statistically significant decrease in blood glucose from 120 min after glucose administration, compared to the negative control (G1) (P<0.05). Therefore, the test substance is considered to be effective for decreasing blood glucose levels.

In the test substance-administered group G4 (1000 mg/kg), a significant decrease in the number of islets (islets/cm²) and islet diameter (m) and a significant increase in zymogen granules %/mm² of exocrine were observed (P<0.01). Accordingly, the test substance, when administered at a dose of 1000 mg/kg, was found to have the effect of repressing morphological changes in pancreatic exocrine and upregulating digestive enzyme secretion and as such, is considered to be effective for treating diabetes.

From the hemato-biochemical test results, it was observed that the test substance-administered group G4 significantly decrease in HbA1c (%) (P<0.05). Thus, the substance is considered to have an effect of reducing blood glucose levels. The test substance-administered group was observed to exhibit a significant decrease in AST (P<0.01) and a decrease trend or a significant decrease in ALT. Hence, the test substance is considered to be a safe substance that does not cause liver dysfunction.

As for blood insulin, its level was observed to have a decrease tendency in the test substance-administered group G3 (750 mg/kg) and significantly decrease in the test substance-administered group G4 (1000 mg/kg). Observation was made of decrease tendency of blood insulin in the test substance-administered groups G3 (750 mg/kg) and G4 (1000 mg/kg), significant decrease tendency of blood c-peptide in the test substance-administered group G3 (750 mg/kg), and a significant decrease of blood c-peptide in the test substance-administered group G4 (1,000 mg/kg), indicating that the test substance reduces insulin tolerance.

As stated in the discussion, the test substance, Irpex lacteus mycelial culture, was considered to be effective for reducing blood glucose as assayed by repeated oral administration for 3 weeks and oral glucose tolerance test (OGTT). The histopathological examination demonstrates the efficacy of the test substance in alleviating diabetes as the test substance-administered group G4 (high dose) was observed to significantly decrease in the number and diameter of pancreatic islets and significantly increase in the proportion of zymogen granules per unit area of exocrine. In addition, a decrease tendency of blood insulin and a significant decrease of c-peptide were observed in the test substance-administered groups G3 (mid dose) and G4 (high dose), demonstrating that the test substance decreases insulin tolerance. For these reasons, a culture of Irpex lacteus is proven to be very effective for preventing and treating diabetes mellitus.

1. (canceled)

2. Irpex lacteus KACC 83046BP.

3. A mycelial culture of Irpex lacteus KACC 83046BP.

4. The mycelial culture of claim 3, wherein the mycelial culture is produced by a culturing method in which the strain is cultured for 5-7 days in a broth with a pH of 5-6 under conditions including an aeration rate of 0.05-2 vvm, a stirring rate of 25-100 rpm, and a culturing temperature of 18-27° C., the broth containing sucrose 0.2-2% (w/v), glucose 0.2-2% (w/v), starch 0.2-2% (w/v), soy flour 0.05-1.5% (w/v), yeast extract 0.05-1.5% (w/v), soy peptone 0.05-1.5% (w/v), MgSO₄ 0.001-0.005% (w/v), KH₂PO₄ 0.001-0.005% (w/v), K₂HPO₄ 0.001-0.005% (w/v), biotin (B7) 0.001-0.005% (w/v), and pyridoxine (B6) 0.001-0.005% (w/v), based on the total volume thereof.

5. A pharmaceutical composition comprising the Irpex lacteus KACC 83046BP of claim 2 or a mycelial culture thereof for prevention or treatment of diabetes mellitus.

6. The pharmaceutical composition of claim 5, having at least one of the functions of reducing a blood bA1c level (%), a blood insulin level, a blood c-peptide level, a blood glucose level, and an OGTT level.

7. The pharmaceutical composition of claim 6, being formulated in at least one form selected from the group consisting of oral dosage forms including pulvis, granules, tablets, capsules, suspensions, emulsions, syrups, and aerosols, topical agents, suppositories, and sterile injectable solutions.

8. A health functional food comprising Irpex lacteus or a mycelial culture thereof for prevention or treatment of diabetes mellitus.

9. A health functional food comprising the Irpex lacteus KACC 83046BP of claim 2 and a mycelial culture thereof for prevention or treatment of diabetes mellitus.

10. The health functional food of claim 9, being in a form of a tablet, a capsule, a pill, an extract, pulvis, granules, a liquid, a suspension, a tea, a jelly, or a beverage.


SEQUENCE LISTING (2 sequences)

SEQ ID NO: 1 - DNA, 1491 bp, Irpex lacteus
tgacaatcagcttgacgggttgtagctggcctctcacgaggcatgtgcacgcctggcttcatccactccttaacctctgtgcactttatgtaagagaaaaaaatggtggaagcttccaggatctcgcgagaggtctttggttgaacaagccgtttttctttcttatgttttactacaaacgcttcagttatagaatgtcaactgtgtataacacatttatatacaactttcagcaacggatctcttggctctcgcatcgatgaagaacgcagcgaaatgcgataagtaatgtgaattgcagaattcagtgaatcatcgaatctttgaacgcaccttgcactccttggtattccgaggagtatgcctgtttgagtctcatggtattctcaacccctaaatttttgtaatgaaggtttagcgggcttggacttggaggttgtgtcggcccttgtcggtcgactcctctgaaatgcattagcgtgaatcttacggatcgccttcagtgtgataattatctgcgctgtggtgttgaagtatttatggtgttcatgcttcgaaccgtctccttgccgagacaatcatttgacaatctgagctcaaatcaggtaggactacccgctgaacttaagcatatcaataaagcggaggaaaggggcaatatcgagttttgaacgggttgtagctggcctctcacgaggcatgtgcacgcctggctcatccctcttaacctctgtgcactttatgtaagaaaaaaaaatggtggaagcttccgggatctcgcgagaggtctttggttgaacaagcgtttttctttcttatgttttactacaacgcttcagttatagaaatgtcactgtgtataaacatttatataccactttcgcaccggaactcttggctctgcatgcattaaaaaaagcacgaaatgccattagtaatggggaattgcaaaattcatggaatcacgaatctttgaagcactggcatcctgggtattccaggaaatatgccggtttgaagtttaggggtattttcaccctaaatttttgttaatgaaaggtttaccgggcttggaacttggaaggttgtggtcgcccttggtcggggcaccccctaaaaaggcattaagcgggaaaactctacagaaacccctccgggggaataaattattttccccctgggggggttttaaaaaaattttttggggggtttagctctccccccccccttccccacaacacttttttaaattttaaacgcggtggggagcccacccccattataaaaatatgggggagaaaaaaaaaaagaccttggtacttgttacccgttgggccgatgcaaaacagcctgatttctggtttagccggcggaagcgtgagggagtaatatgaatggcgcacgatagaccaaacccacccgcccgcctgacttacggaggcccgccgccttaccggcacggccatgggggtgttatggtgcgaagtaagg

SEQ ID NO: 2 - DNA, 1209 bp, Irpex lacteus
gaaattataaaatttttccggtgcgcattggtgcactagtaaggtgttcttttttaatgcgacccgcttttcttttaaggttgatgaatgacattaagtgtaaggactacttgatcaatggttacgaacggattacgcgattggacctgctttcctatgacctactacccacgccacttcaaaatatttatagtacgagaaacgtccactactttctattttcgtttaacaacggatcgcttggctccccgatcaaagacgagggtaacagcgcggcataactagtgcgaattgtatatgattcccttcattcagttgttcaccccatctagtcctcgttggtccgccctccatcacgcctggaccgcacaaccgctgcccccctcattattatatgggggatgcacctgggaaattctataagtgaggagtctgtccctaacatgaaaaaaaaaaatgggctgggcgcccgaatacctctcccgcgatcccggaggctttccccattttttttttctctgaaaactgcacaaagaagattagtggatgatgcccccggggcgatgtcgcttgagaggagaacttctaccccctctaaaaccccttgaggggccttctaggttttcctcggaaggaatcattatcgagttttgaacgggttgtagctggctctcacgaggcatgtgcacgcctggctcatcactcttaactctgtgcactttatgtaagagaaaaaaatggtggaagcttcaggatctcgcgagaggtctttggttgaacaagcgtttttctttcttatgttttactacaacgcttcagttatagaatgtcactgtgtatacaatttatatacactttcagcacggatctcttggctctcgcatcgattaagaacgcacgaaatgcgataagtaatgtgaattgcagaaattcgtgaatcatcgaatcttttgaacgcacttgcatcttgggtattcagggagtatgctgcttgactctcaggtatttccaccttaaatttttgtatgagatttacgggctggacttgaggatgggtcgcttgtcgtcactccttgaaatgcattaagtgattacgatgctcaggtgatattatctgccttggatgagaattatgggttaggctacgatcttgcgaaacttggacgtactgctagagctactacgctattgatagtgggagaacaagga